(19) United States
(12) Patent Application Publication
(10) Pub. No.: US 2016/0287506 A1
(43) Pub. Date: Oct. 6, 2016

(54) Hair Treatment Compositions

(71) Applicant: ELC MANAGEMENT LLC, Melville, NY (US)

(72) Inventors: Wilson A. Lee, Hauppauge, NY (US); George J. Stepniewski, Melville, NY (US)

(21) Appl. No.: 15/075,801

(22) Filed: Mar. 21, 2016

Related U.S. Application Data
(60) Provisional application No. 62/142,729, filed on Apr. 3, 2015.

(51) Int. Cl. A61K 8/64; A61K 8/88; A61Q 1/10; A61K 8/81

(52) U.S. Cl. CPC A61K 8/645; A61K 8/817; A61K 8/88; A61K 8/8152; A61Q 1/10; A61K 2800/43; A61K 2800/884

(57) ABSTRACT

Hair treatment compositions comprising an aqueous base, hydrolyzed wheat protein PG-propyl silanetriol, Polyquaternium-6, and, optionally, polylysine and/or an acrylates-based film former.

### FIELD OF THE INVENTION

The invention pertains to a type of charged protein complex that may be applied to hair of the head prior to applying makeup or other treatment, or that may be applied to hair simultaneously with makeup or other treatment product. In one embodiment, the invention is directed to a product for the eyelashes that may be used as a pretreatment product or as a full formula mascara.

### BACKGROUND

Mascara is used for coloring and grooming the eyelashes to make the lashes appear fuller and longer. For the most part, mascara products try to achieve these objectives by applying a thick coating of viscous material to the lashes. These conventional lash grooming products are temporary, which is to say that they wash off relatively easily in a single washing, and no long term change to the structure of the eyelashes is achieved. It would be beneficial if, once applied, an eyelash product could deliver a long term benefit that does not wash out with several washings. The present invention provides such a benefit.

It is well known that life cycle of the eyelash is approximately 90 days, which is 8-24 times shorter than that of scalp hair. The frequent use of aqueous and/or waterproof mascaras can dry eyelashes, especially if the formulation does not contain moisturizers, conditioners or humectants. In fact, a positive correlation has been demonstrated between the frequency of mascara use and the degree of cracking in hair cuticles, the thickness of the cuticle, and the porosity in cortex. (see, “Internal structure changes of eyelash induced by eye makeup,” K. Fukami, T. Inoue, T. Kawai, K. Sakata, M. Nagano, K. Takehara). These correlations demonstrate that mascara or mascara remover can induce cracks or swelling in the cuticle of the hair and increase the porosity of cortex. The result is dry, weakened hair.

Likewise, hair on the top of the head is subject to degradation from various factors, including frequent washing, harsh chemical treatments, harsh heat treatments, air pollution, UV exposure, and other factors. Swollen and cracked cuticles and increased porosity tends to make hair dry, limp and unmanageable, and induces split ends and loss of luster. Other body hair, such as eyebrow hair may suffer from some of the same problems.

Proteins, such as hydrolyzed wheat and vegetable proteins, and amino acids have been used in hair care products as treatment for damaged hair. Wheat proteins are known to have good substantivity to hair, while reportedly penetrating the cuticle to provide conditioning benefits and improve break strength. Despite their substantivity, proteins deposited on the hair are susceptible to being rinsed away after one or more washings, along with any benefit that they might confer. The present invention enhances the substantivity of certain hydrolyzed wheat proteins and amino acids, and extends the benefits thereof. Thus, the present invention offers significantly greater protection from damage caused by known eyelash products.

Stable colloidal dispersions are characterized by an electrical double layer at the interface between the surface of the colloid and the bulk fluid dispersion medium. We sometimes speak of a “slipping plane” as a surface that surrounds the colloid. Inside the slipping plane, molecules of the bulk fluid are attached to the colloid surface. Outside the slipping plane, the bulk fluid remains mobile. Zeta potential (ζ-potential) refers to the difference in electric potential between the slipping plane of the double layer interface and a location in the bulk fluid away from the interface. The magnitude of the ζ-potential is a measure of the repulsive force between similarly charged colloid particles, and thus, provides a measure of the stability of a colloidal dispersion. Generally, a higher ζ-potential indicates greater stability. In the present invention, ζ-potential may aid in the identification of those proteins that are more suitable to forming the types of protein complexes that adhere to the eyelashes (or other hair).

### SUMMARY

We have invented charged protein-polymer complexes that bind to human hair, be it hair of the head, eyebrows or eyelashes. In one embodiment, the complex is particularly useful for making up the eyelashes and protecting the eyelashes from harsh treatments, such as washing. In other embodiments, the complex can be delivered from a conditioner, shampoo or other hair treatment product. The complex is formed by reacting a cationic polymer with hydrolyzed wheat protein PG-propyl silanetriol. The charged protein-polymer complex can be delivered to the hair as a pre-treatment prior to applying a hair care or hair treatment product. Alternatively, the complex may be incorporated into a hair care or makeup composition, such as a mascara, shampoo or conditioner for simultaneous application to the hair. Once deposited on the hair, the protein-polymer complex does not wash or rinse out after several attempts, even when the post-applied or simultaneously-applied hair product is washed away. Some embodiments of the invention include polylysine and/or one or more film forming agents that further enhance the protein complex. The protein-polymer complexes have the effect of repairing and/or protecting the cuticles of the hair from damage associated with use of makeup or treatment products, treatment regimens and environmental aggressors.

### DETAILED DESCRIPTION

All percentages mentioned herein are percentages by weight of the total composition, unless otherwise indicated. Without loss of generality, the following discussion focuses on mascara and eyelashes.

The word “comprise” and its derivatives means that a group of items is not limited to those explicitly recited, and may or may not contain more items.

The surface of human hair is, naturally, negatively charged. Thus, in general, positively charged proteins will bind more readily to hair than neutral or negatively proteins. The approach taken here was to form a complex of a positively charged cationic polymer and a hair beneficial protein or a hair beneficial amino acid, or both.

The task was to find useful combinations of hair beneficial proteins/amino acids and cationic polymers that, once applied, adhere to the hair and protect the integrity of the cuticle and the cortex. Other considerations include being able to disperse the protein-cationic polymer complex in delivery vehicle that has a pH that is acceptable for use on the eyelashes (typically about 6.5-8.0), while maintaining commercially acceptable shelf-life stability of the vehicle. The first stage of our research consisted of several proof of concept experiments. After elimination several potential ingredients, we focused on the following materials.

Hydrotriticum™ 2000 PE from Croda, Inc. is a 20% aqueous solution of hydrolyzed wheat proteins and amino acids having an average molecular weight of about 3,000 Daltons in a range of about 75 to 10,000 Daltons; CAS-No. 70084-87-6).

Crodasone™ W PF-LQ-WD from Croda, Inc. is a 26-30% aqueous solution of the polymer whose chemical name is protein hydrosylates, wheat, [2-hydroxy-3-[3-trimethoxysilyl)propoxy]propyl], hydrolyzed (CAS-No. 152887-30-4). The polymer is also known as hydrolyzed wheat protein PG-propyl silanetriol.

Quinoa from Vege Tech Company (Glendale, Calif.); range of molecular weights from 300-200,000 Daltons.

Merquat™ 100 is a 39-44% aqueous solution of Polyquaternium-6 (a highly charged cationic homopolymer of diallyl dimethyl ammonium chloride); available from Lubrizol Corporation.

A 25% solution of polylysine was sometimes used as a pH adjuster.

Experiment 1: To verify that a complex of protein/amino acids and a cationic polymer possess better substantivity than the protein or cationic polymer alone.
1. Prepared the four solutions shown in the following table, with propeller mixing. A1 is a control solution. Polylysine was used as a pH adjuster.
2. Prepared 4 bundles of gray hairs; soaked them in control solution for 3 hours; allowed bundles to dry at 50° C.
3. Immersed 1 bundle in each of the solutions X1, P1 and C1, for 4 hours; allowed them to dry at 50° C.
4. On all four bundles, measured L*a*b* values at three different points using a spectrophotometer.
5. Washed each hair bundle in a jar of water, with shaking; repeated to 30 times; allowed to dry at 50° C.
6. Measured L*a*b* values at three different points using a spectrophotometer, and calculated ΔE for each sample according to:

ΔE=Sqrt {(L₁−L₂)²+(a₁−a₂)²+(b₁−b₂)²}.

The results are shown in the following table.

| | X1 | P1 | C1 | A1 |
|---|---|---|---|---|
| | % Concentration | | | |
| Protein | | | | |
| Crodasone ™ W PF-LQ-WD | 25 | 21.14 | | |
| Amino Acid | | | | |
| Polylysine 25% solution | 3.0 | | | |
| Cationic Polymer | | | | |
| Polyquaternium-6 | 1.56 | | 21.14 | |
| water | 70.34 | 78.76 | 78.76 | 99.9 |
| Red 33 | 0.1 | 0.1 | 0.1 | 0.1 |
| pH | 7.29 | 4.95 | 8.23 | |
| ζ-potential (millivolts) | 28.95 | −8.2 | 51.49 | |
| ΔE after water rinsing | 11.01 | 63.73 | 176.78 | 224.59 |
| ΔE after rinsing with | 6.15 | 108.19 | 60.86 | 130.36 |
| hydrocarbon | | | | |
| Permethyl ® 99A | | | | |

A relatively large ΔE indicates that the color of the hair sample has changed a lot, due to the red dye being rinsed out of the hair. A relatively small ΔE indicates that the color of the hair sample has changed little, due to the red dye color being retained in the hair, even after repeated washing. When a test sample retains more color than the control, we attribute the retention of color to the retention of the protein, which repairs damage to the cuticle.

By far, the X1 sample, with a complex of hydrolyzed wheat protein and cationic polymer, performed the best (lowest ΔE), significantly better than sample P1, having the hydrolyzed wheat protein, but no cationic polymer, and significantly better than the cationic polymer alone. The entire experiment was repeated with hydrocarbon washing, and sample X1 again performed better by far.

We theorize that the cationic polymer is improving the substantivity of the hydrolyzed wheat protein PG-propyl silanetriol, which translates into superior cuticle repair that remains even after repeated water or hydrocarbon washing. Furthermore, the improvement in substantivity seems to be synergistic, more improvement than could be expected from the individual contributions of the sum of protein and cationic polymer.

Experiment 2: To further investigate hydrolyzed wheat protein PG-propyl silanetriol (Crodasone™ W PF-LQ-WD), as well as two other vegetable proteins for their suitability in commercial, cosmetic eye products.

### Step I:

Blend different ratios of protein and Polyquaternium-6 in solution, attempt to adjust pH with polylysine, and note the stability.

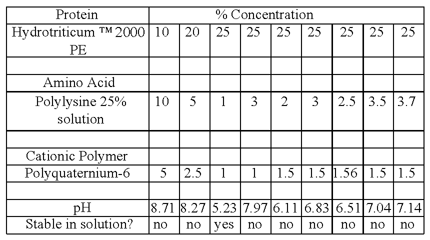

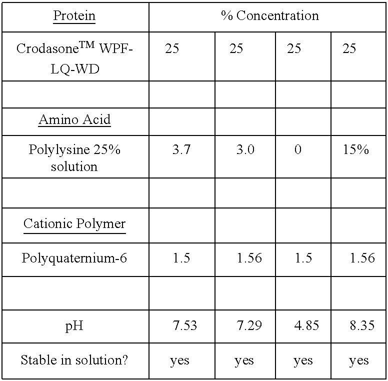

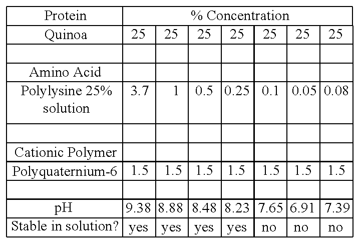

### Step 2: Determine ΔE

From Step 1, select the shaded solutions for further study. All of these were prepared in aqueous solution with 0.1% by weight of Red 33; q.s. water to 100. Also, a 0.1% aqueous solution of Red 33 was used as a control (labeled A1).

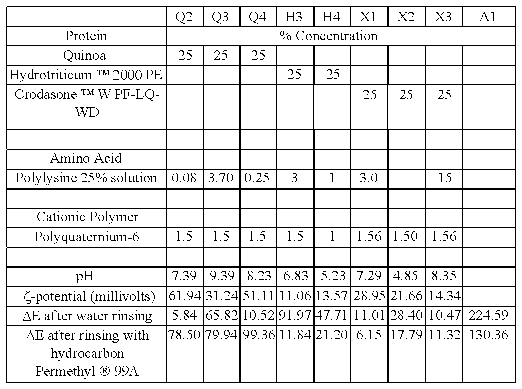

1. Prepared 9 bundles of gray hairs; soaked them in the control solution for 3 hours; allowed bundles to dry at 50° C.
2. Immersed 1 bundle in each of the solutions above, for 4 hours; allowed them to dry at 50° C.
3. Measured L*a*b* values at three different points using a spectrophotometer.
4. Washed each hair bundle in a jar of water, with shaking; repeated to 30 times; allowed to dry at 50° C.
5. Measured L*a*b* values at three different points using a spectrophotometer, and calculated AE for each sample:

ΔE=Sqrt{(L₁−L₂)²+(a₁−a₂)²+(b₁−b₂)²}.

A relatively large AE indicates that the color of the hair sample has changed a lot, due to the red dye being rinsed out of the hair. A relatively small ΔE indicates that the color of the hair sample has changed little, due to the red dye color being retained in the hair, even after repeated washing. When a test sample retains more color than the control, we attribute the retention of color to the retention of the protein complex, which repairs damage to the cuticle.

The results are shown in the table above. In this preliminary study, the sample treated with Q2 complex (Quinoa) had the lowest ΔE (5.84) after rinsing with water, which means that the hair had a relatively high substantivity for the Q2 complex, at least in an aqueous environment. However, when the entire experiment was repeated with hydrocarbon washing, sample Q2 did not fair so well. The Q4 Quinoa sample performed similarly, well against water rinsing, but not against hydrocarbon rinsing. The Q3 Quinoa sample did not perform well with either hydrocarbon or water rinsing. In total, this means that the Quinoa complex is not resistant to hydrocarbons, which indicates that the complex could also be removed from the hair by sebum oil, in which case the cuticle may not benefit to a significant degree.

In contrast, the Hydrotriticum™ 2000 PE samples performed well in hydrocarbon rinsing, but not in water rinsing. This means that this specific wheat protein complex will not be removed from the hair by sebum oil, but will be removed by moisture. While not ideal, this hydrolyzed wheat protein complex offers some benefit to hair in between rinsings, or as art of an anhydrous system.

The best performing complexes were X1 and X3 (hydrolyzed wheat protein PG-propyl silanetriol (Crodasone™ W PF-LQ-WD) with pH adjusted by polylysine), which had some of the lowest ΔE values after rinsing with water or hydrocarbon. For water rinsing, the X1 and X3 complexes performed about 20 and 21 times better, respectively, than the control sample, A1. For hydrocarbon rinsing, the X1 and X3 complexes performed about 21 and 12 times better, respectively, than the control sample, A1. This indicates that these complexes provide a beneficial amount of cuticle repair, even after water and hydrocarbon washing. Interestingly, the X2 complex (Crodasone™ W PF-LQ-WD without polylysine, i.e., low pH) did not perform as well, but still showed significant improvement over the control and was overall better than the Hydrotriticum™ 2000 PE and quinoa samples. We note that the pH of the X2 sample (4.85) is low compared to X1 (7.29) and X3 (8.35), and this may account for X2 not performing as well.

So far, it can be said that the hydrolyzed wheat protein samples are performing significantly better than the quinoa protein samples. Also, the hydrolyzed wheat protein PG-propyl silanetriol (Crodasone™ W PF-LQ-WD) samples are performing better than the Hydrotriticum™ 2000 PE protein samples. Quinoa and Hydrotriticum™ 2000 PE contain cysteine, which allows them to covalently bond to hair through disulphide bonding.

Crodasone™ W PF-LQ-WD is a polysiloxane. It is a 26-30% aqueous solution of protein hydrosylates, wheat, [2-hydroxy-3-[3-trimethoxysilyl)propoxy]propyl], hydrolyzed. The solids portion of Crodasone™ W PF-LQ-WD is reportedly 71.4% wheat proteins and 28.6% of 3-glycidoxypropyl trimethoxysilane. The 3-glycidoxypropyl trimethoxysilane (GPTMS) reacts with the side chain or end terminal amino groups of the peptide chains in the hydrolyzed wheat protein to produce the protein silicone polymer. Any residual GPTMS readily hydrolyses to give 2,3-hydroxypropyl trihydroxysilane, which then polymerizes to form a polysilicone. Without offering an explanation, we merely note that the hydrolyzed wheat protein in polysiloxane form (i.e. Crodasone™ W PF-LQ-WD) is giving better results than the hydrolyzed wheat proteins that are capable of disulphide bonding (i.e. Quinoa and Hydrotriticum™ 2000 PE). This is unexpected.

Furthermore, regarding the hydrocarbon rinse test, the three samples that performed well (X1, X3, H3) had a pH from 6.83-8.35 and a ζ-potential between about 11-30 mV. The next two that performed best (X2, H4) had ζ-potentials between about 13 and 22 mV, but lower pH (4.85 and 5.23). The worst three results were achieved from samples that had pH 7.39-9.39 (which overlaps the pH of the best performers), but ζ-potentials greater than 30 mV (which is outside of the range of the ζ-potentials of the best performers).

Regarding the water rinse test, the four samples that performed well (X1, X3, Q2, Q4) had a pH from 7.29-8.35 and a ζ-potential between about 14-62 mV. The next two that performed best (X2, H4) had ζ-potentials between about 13 and 22 mV, but lower pH (4.85 and 5.23). The worst two results were achieved from samples that had ζ-potentials of about 11 and 31 mV (which overlaps the -potentials of the best performer), but pH of 6.83 and 9.39 (which is outside of the range of the pH of the best performers).

Overall, these results suggest that good results in the water rinse test and the hydrocarbon rinse test are expected when the protein and cationic polymer complex is delivered in a vehicle that has a pH of about 7.3-8.5 and a ζ-potential of about 10-30 mV. Furthermore, polylysine, which was originally added to adjust pH, seems to have a beneficial effect on the hydrolyzed wheat protein samples, increasing the hair's retention of the wheat proteins, but a generally worsening effect on the quinoa protein samples.

Experiment 3: To investigate the impact of applying an oil-in-water mascara emulsion on top of the X1 complex (hydrolyzed wheat protein PG-propyl silanetriol and Polyquaternium-6).
1. Prepared the X1 solution, as described above. A1 is a control solution.
2. Prepared 4 bundles of gray hairs; soaked them in control solution for 3 hours; allowed bundles to dry at 50° C.
3. Immersed the 3 bundles in solution X1 for 4 hours; allowed them to dry at 50° C.
4. On all three bundles, measured L*a*b* values at three different points using a spectrophotometer.
5. Apply an oil-in-water mascara (Estee Lauder High Impact Mascara) to each bundle; allowed bundles to dry at 50° C.
6. Washed each hair bundle in a jar of 50% water, 50% Permethyl® 99A, with shaking; repeated to 30 times; allowed to dry at 50° C.
7. Measured L*a*b* values at three different points using a spectrophotometer, and calculated ΔE for each sample.

The average ΔE for the X1 test sample with oil-in-water mascara was 244. This result shows that the oil-in-water mascara is interfering with the adhesion of the protein complex. We suspected that the emulsifiers of the oil-in-water mascara are having a detrimental effect on the adhesion property of protein-cationic polymer complex.

We repeated the same experiment as above with an anhydrous mascara that comprises suspending agents, hair conditioning agents, pigment and preservative, but no emulsifiers. The average ΔE for the X1 test sample with the anhydrous mascara was 12.41, compared to 85.05 for a control sample. This is a clear indication that the emulsifiers of the oil-in-water mascara were interfering with the adhesion of the protein complex, while the result with the anhydrous mascara shows that the benefit of protein adhesion to the eyelashes can be realized in a real life use situation. For example, an aqueous solution of a hydrolyzed wheat protein-cationic polymer complex can be used for a real benefit in a two-regimen product. In a pretreatment step, the eyelashes are treated with an aqueous solution of a protein-cationic polymer complex such as X1, herein, and then an anhydrous mascara is applied over that. Even when the mascara is rinsed out, a significant amount of the protein remains adhered to the hair, repairing the hair and alleviating the damage caused by washing out the anhydrous mascara.

Experiment 4: To investigate the impact of applying an oil-in-water mascara emulsion on top of the X1 complex modified by a film former.

Based on the previous results with oil-in-water mascara, we thought that we could improve protein adhesion to the lashes if we added a film former to the aqueous solution of protein-polylysine-cationic polymer complex. Various PVP-, polyurethane-, acrylate- and acetate-type film formers were tried. Excellent results were Syntran® PC5776 (Interpolymer Corp.), which has good durability against water and oil after it is dried. The Experiment above was repeated, except that formula X1 was replaced by formula X11, as shown:

X11

| | |
|---|---|
| Crodasone ™ W PF-LQ-WD | 25.00 |
| Polylysine 25% solution | 3.00 |
| Polyquaternium-6 | 1.56 |
| water | 69.34 |
| Red 33 | 0.10 |
| ^{a}Syntran ® PC5776 | 1.00 |
| pH | 7.34 |

^{a}water/acrylates/ethylhexyl acrylate/hema/styrene copolymer/acrylates/dimethylaminoethyl methacrylate

The average ΔE for the X11 test sample with oil-in-water mascara was 6.04, a dramatic improvement over the X1 test sample without film former. Thus, the film former, Syntran® PC5776, protected the protein-polylysine-cationic polymer complex from interference with the emulsifiers in the oil-in-water mascara.

This result further suggests that the aqueous solution of a hydrolyzed wheat protein PG-propyl silanetriol-Polyquaternium-6 complex can be used for a real benefit in a two-regimen product. In a pretreatment step, the eyelashes are treated an aqueous solution of hydrolyzed wheat protein PG-propyl silanetriol-Polyquaternium-6 complex, such as X11, herein, and then an oil-in-water mascara is applied over that. Even when the mascara is rinsed out, a significant amount of the protein remains adhered to the hair, repairing the hair and alleviating the damage caused by washing out the oil-in-water mascara.

We repeated the same experiment as above with an anhydrous mascara that comprises suspending agents, hair conditioning agents, pigment and preservative, but no emulsifiers. The average ΔE for the X11 test sample with the anhydrous mascara was 9.55, compared to 12.41 for the X1 sample. Thus, the film former has also improved the adhesion of the hydrolyzed wheat protein PG-propyl silanetriol-Polyquaternium-6 complex when the complex is used as a pretreatment step before applying an anhydrous mascara.

Based on our observations, a useful composition will comprise hydrolyzed wheat protein PG-propyl silanetriol in the range of 0.0025 to 15% by weight of the composition. If the hydrolyzed wheat protein is provided in the form of Crodasone™ W PF-LQ-WD, then the range would be about 0.01 to about 50% by weight.

A useful range of Polyquaternium-6 is 0.004 to 6% by weight of the composition. If the Polyquaternium-6 is Merquat™ 100, then a useful range is about 0.01% to 15% by weight of the composition.

Polylysine is optional, and seems to enhance the retention of proteins on the hair. If used, a useful range of polylysine is 0.001 to 4% by weight of the composition.

If used, acrylates-based film former (such as Syntran® PC5776) may comprise from 0.5%-15% by weight of the composition.

The remainder of the composition is a cosmetically acceptable aqueous base that comprises 50% to 80% of water by weight of the composition, and cosmetically acceptable adjuvants that are commonly used in hair products, such as colorants, moisturizers, thickeners and pH adjusters.

An example of an effective eyelash pretreatment composition according to the present invention is shown in column A of the following table.

| | A | B |
|---|---|---|
| hydroxyethycellulose | 1.8 | 1.8 |
| potato starch | 0.5 | 0.5 |
| propanediol | 2.0 | 2.0 |
| phenoxyethanol | 0.4 | 0.4 |
| black iron oxides | — | 3.0 |
| denatured alcohol | 10 | 10 |
| seaweed extract | 2.0 | 2.0 |
| ^{a}Syntran ® PC5776 | 10 | 10 |
| Polylysine 25% solution | 0.15 | 0.15 |
| ^{b}Merquat ™ 100 | 0.35 | 0.35 |
| ^{c}Crodasone ™ W PF-LQ-WD | 4.5 | 4.5 |
| water | Q.S. | Q.S. |

^{a}water/acrylates/ethylhexylacrylate/hema/styrene copolymer/acrylates/dimethylaminoethyl methacrylate
^{b}39-44% aqueous solution of Polyquaternium-6
^{c}26-30% aqueous solution of hydrolyzed wheat protein, [2-hydroxy-3-[3-trimethoxysilyl)propoxy]propyl], hydrolyzed.

The formula of column B demonstrates that an eyelash pretreatment composition according to the present invention may comprise colorants, such as black iron oxides. When this is the case, the composition may still be formulated as a pretreatment composition which provides color to the eyelashes in addition to the color supplied by a mascara composition that is applied over the pretreatment composition. Alternatively, the formula of column B is a complete, commercially viable mascara product. When this formula was applied to the eyelashes with a typical mascara brush, very good lash separation and lengthening was achieved. When the eyelashes were washed, color was removed, but protein-cationic polymer complex remained on the eyelashes.

The compositions in columns A and B have a pH of 7.2, and are commercially stable. Thus, compositions of the invention may serve as pretreatment compositions for the eyelashes (and any other hair of the body) or as finished products, such as mascara, shampoo, conditioner, and eyebrow makeup. Any of these product types may include pigments or dyes. The most suitable pigments or dyes will be hydrophilic, either naturally or surface treated to be so. One or more hydrophilic pigments or dyes may be included in compositions of the invention at 0.001-12% by weight of the composition.

The present invention also covers methods of treating the eyelashes. In a first method, a pretreatment composition according to the present invention having no acrylates based film former is provided, and is applied to the eyelashes; the pretreatment composition is allowed to dry for at least one minute, preferably at least two minutes; then an anhydrous mascara composition is applied to the eyelashes, over the pretreatment composition.

In a second method, a pretreatment composition according to the present invention having acrylates based film former, as described herein, is provided, and is applied to the eyelashes; the pretreatment composition is allowed to dry for at least one minute, preferably at least two minutes; then a water based or anhydrous mascara composition is applied to the eyelashes, over the pretreatment composition.

In a third method, a composition of the invention that includes pigment or dyes is provided, and the composition is applied to the eyelashes.

In a fourth method, a composition of the invention is provided, the composition is applied to hair, and subsequently rinsed out of the hair with water. For example, a shampoo, conditioner or eyebrow product comprising hydrolyzed wheat protein PG-propyl silanetriol-Polyquaternium-6 complex according to the invention is provided, and the composition is applied to the hair of the head or eyebrows, and subsequently rinsed out of the hair with water.

What is claimed is:

1. A hair treatment composition comprising:
A cosmetically acceptable aqueous base;
hydrolyzed wheat protein in a range of 0.0025 to 15% by weight of the composition; and
Polyquaternium-6 in a range of 0.004 to 6% by weight of the composition.

2. The composition of claim 1 wherein the hydrolyzed wheat protein is hydrolyzed wheat protein, 2-hydroxy-3-[3-trimethoxysilyl)propoxy]propyll hydrolyzed.

3. The composition according to claim 2, further comprising polylysine from 0.001% -4% by weight of the composition.

4. The composition according to claim 2, further comprising an acrylates-based film former from 0.5%-15% by weight of the composition.

5. The composition according to claim 4, wherein the acrylates-based film former is acrylates/ethylhexylacrylate/hema/styrene copolymer/acrylates/dimethylaminoethyl methacrylate film former.

6. The composition according to claim 2, further comprising 0.001 to 12% of hydrophilic pigment or dye, by weight of the composition.

7. A method of treating hair of the body comprising the steps of:
providing a composition according to claim 3;
applying the composition to the hair;
rinsing the composition out of the hair with water.

8. A method of treating eyelashes comprising the steps of:
providing a first composition according to claim 2;
applying the first composition to the eyelashes;
allowing the first composition to dry on the eyelashes for at least one minute; and
applying to the eyelashes an anhydrous mascara composition over the first composition.

9. A method of treating eyelashes comprising the steps of:
providing a first composition according to claim 4;
applying the first composition to the eyelashes;
allowing the first composition to dry for at least one minute; and
applying to the eyelashes a water-based mascara composition over the first composition.

10. A method of treating eyelashes comprising the step of applying to the eyelashes a composition according to claim 6.
